(19) United States
(12) Patent Application Publication
(10) Pub. No.: US 2025/0195492 A1
(43) Pub. Date: Jun. 19, 2025

(54) USE OF IPTACOPAN FOR THE TREATMENT OF LUPUS NEPHRITIS

(71) Applicant: Novartis AG, Basel (CH)

(72) Inventors: Janina LINNIK, Zurich (CH); Matthias MEIER, Grenzach-Wyhlen (DE); Nicholas WEBB, Basel (CH)

(21) Appl. No.: 18/843,599

(22) PCT Filed: Mar. 3, 2023
(86) PCT No.: PCT/IB2023/052010

§ 371 Date: Sep. 3, 2024

Related U.S. Application Data
(60) Provisional application No. 63/316,623, filed on Mar. 4, 2022.

(51) Int. Cl. A61K 31/454; A61K 45/06

(52) U.S. Cl. CPC A61K 31/454; A61K 45/06

(57) ABSTRACT

Described herein are methods of treating lupus nephritis with the Factor B inhibitor iptacopan or a pharmaceutically acceptable salt thereof, e.g. iptacopan hydrochloride.

### FIELD

The disclosure relates to methods of treating complement driven diseases, and in particular, lupus nephritis (LN) with the Factor B inhibitor iptacopan or a pharmaceutically acceptable salt thereof, e.g. iptacopan hydrochloride.

### BACKGROUND

Systemic lupus erythematosus (SLE) is a group of autoimmune diseases of diverse causes sharing a loss of immune tolerance of endogenous nuclear material, which leads to systemic autoimmunity that can lead to damage to various tissues and organs. Lupus nephritis (LN) is a form of glomerulonephritis and constitutes one of the most severe organ manifestations of SLE: 60% of adult patients with SLE develop kidney disease (Anders et al 2020, Lupus nephritis. Nat Rev Dis Primers p. 7), which represents a major cause of morbidity and mortality (Fanouriakis et al 2020, 2019 Update of the Joint European League Against Rheumatism and European Renal Association-European Dialysis and Transplant Association (EULAR/ERA-EDTA) recommendations for the management of lupus nephritis. Ann Rheum Dis p. 713-723).

Lupus nephritis is caused by loss of tolerance to self-antigens, the production of autoantibodies and deposition of complement-fixing immune complexes (ICs). IC-mediated activation of complement in affected tissues is evident in both experimental and human SLE along with pathological features that are logical consequences of complement activation (Bao et al 2015, Complement in Lupus Nephritis: New Perspectives. Kidney Dis (Basel) p. 91-9). The activation of the alternative complement pathway of the innate immune system plays an important role in the pathogenesis of LN (Song et al 2017, Complement Alternative Pathway's Activation in Patients With Lupus Nephritis. Am J Med Sci p. 247-257). Immune complex formation in LN is related to a plethora of autoantibodies, especially anti-dsDNA and anti-nucleosome antibodies (ANA), is the result of systemic autoimmunity and is a hallmark of the disease (Waldman and Madaio 2005, Pathogenic autoantibodies in lupus nephritis. Lupus p. 19-24: Nowling and Gilkeson 2011, Mechanisms of tissue injury in lupus nephritis. Arthritis Res Ther p. 250).

The pathophysiology of LN is heterogeneous. Genetic and environmental factors likely contribute to this heterogeneity. Despite improved understanding of the pathogenesis of LN, treatment advances have been few and risk for kidney failure remains unacceptably high (Parikh et al 2020, Update on Lupus Nephritis: Core Curriculum 2020. Am J Kidney Dis p. 265-281). As a bridge between the innate and adaptive immune systems, the complement system is likely to participate in the multiple processes of pathogenesis in LN. LN is categorized histologically into 6 classes by the international Society of Nephrology/Renal Pathology Society (ISN/RPS) classification system (Markowitz and D'Agati 2007, The ISN/RPS 2003 classification of lupus nephritis: an assessment at 3 years. Kidney Int p. 491-5). Moderate to severe (Classes III and IV) LN are detected in approximately 39 to 71.9% of patients and have deposition of ICs in the subendothelial space of the glomerular capillaries (Wang et al 2018, A Systematic Review and Meta-analysis of Prevalence of Biopsy-Proven Lupus Nephritis. Arch Rheumatol p. 17-25). Both these classes of LN are considered to have similar lesions that differ by severity and distribution. Class IV diffuse LN can be distinguished from class III based on involvement of more than 50% of glomeruli with endo-capillary lesions. The prognosis of LN depends on the histological classification, the degree of active inflammation and the chronic interstitial damage. LN patients with ISN/RPS class III-IV are at a greater risk of loss of kidney function and the development of kidney failure.

Treatment for LN is dependent on disease severity and patient variables, based on histopathological and/or clinical manifestations (Flanc et al 2004, Treatment for lupus nephritis. Cochrane Database Syst Rev p. CD002922). Treatment usually consists of intense immunosuppressive induction therapy for a 3 to 6-month period to induce complete or at least partial remission, which is followed by long-term, less aggressive, maintenance therapy to maintain disease remission and to prevent disease flares. The definition of a complete response can vary considerably from study to study (Boumpas and Balow 1998, Outcome criteria for lupus nephritis trials: a critical overview. Lupus p. 622-9). Complete remission is defined as a normalization in proteinuria and serum creatinine, patients who attained a complete remission with aggressive immunosuppressive treatment had significantly better patient and renal survivals than nonresponders (Korbet et al 2000, Factors predictive of outcome in severe lupus nephritis. Lupus Nephritis Collaborative Study Group. Am J Kidney Dis p. 904-14).

The American College of Rheumatology (ACR) Guidelines (Hahn et al 2012, American College of Rheumatology guidelines for screening, treatment, and management of lupus nephritis. Arthritis Care Res (Hoboken) p. 797-808) and the joint European League Against Rheumatism (EULAR)/European Renal Association-European Dialysis and Transplant Association (ERA-EDTA) and KDIGO (Shlipak et al 2021, The case for early identification and intervention of chronic kidney disease: conclusions from a Kidney Disease: Improving Global Outcomes (KDIGO) Controversies Conference. Kidney Int p. 34-47) guidelines are uniform in their recommendations for therapy for class III and IV LN and include a sequence of induction and maintenance phases. Goals of treatment include patient survival, long-term preservation of kidney function, prevention of disease flares, prevention of organ damage, management of comorbidities and improvement in disease-related quality of life. LN patients with active Class III/IV±V LN or pure Class V LN with heavy proteinuria should begin treatment with combination immunosuppression, including high-dose corticosteroids and cyclophosphamide (CYC), mycophenolate mofetil (MMF) or mycophenolate sodium (MPS) (Yap and Chan 2015, Lupus Nephritis in Asia: Clinical Features and Management. Kidney Dis (Basel) p. 100-9). The primary adjunct therapies in the treatment of LN include hydroxychloroquine (HCQ) (unless contra-indication), ACEi/ARB, strict BP control (<130/80 mmHg), statins (ACR recommendations 2012) dietary sodium restriction, vitamin D repletion, weight loss and correction of metabolic abnormalities (acidosis, hyperuricaemia).

LN treatment requires an initial intensive period of therapy followed by a long-term maintenance treatment period in order to stabilize the disease and ultimately reach renal remission (Moroni et al 2018, Changing patterns in clinical-histological presentation and renal outcome over the last five decades in a cohort of 499 patients with lupus nephritis. Ann Rheum Dis p. 1318-1325). An important challenge in the management of LN is the continued use of corticosteriods for longer periods (Little J, Parker B, Lunt M, et al (2018) Glucocorticoid use and factors associated with variability in this use in the Systemic Lupus International Collaborating Clinics Inception Cohort. Rheumatology (Oxford) p. 677-687). Chronic corticosteriod therapy is associated with short and long-term adverse events (Anders, H J., Saxena, R., Zhao, Mh. et al. Lupus nephritis. Nature Review Disease Primers 6, 7 (2020)). The optimal treatment of LN remains a challenge despite of available treatment have improved outcomes.

The complement alternative pathway (AP) is important for innate and adaptive immunity. However, hyperactivity of AP is known to cause and worsen a wide number of diseases with autoimmune components. Iptacopan is a novel oral small molecular weight compound, with first in class potential, that inhibits factor B (FB) of the AP. The AP amplifies complement activation induced by any complement pathway and results in significant decrease of C3 and increase of Bb, C3a, C5a and MAC in active LN (Ekdahl et al 2018, Interpretation of Serological Complement Biomarkers in Disease. Front Immunol p. 2237).

Iptacopan is a first-in-class, oral, low molecular weight (LMW) inhibitor of Factor B (FB) (Schubart et al 2019, Small-molecule factor B inhibitor for the treatment of complement-mediated diseases. Proc Natl Acad Sci USA p. 7926-7931), a key protease of the AP (Merle et al 2015, Complement System Part I—Molecular Mechanisms of Activation and Regulation. Front Immunol p. 262). Inhibition of FB prevents amplification of all pathways as well as AP-induced assembly of C3- and C5-convertases. At the same time, iptacopan has only limited effect on classical-pathway induced activation of the terminal pathway. Iptacopan inhibits FB in the context of the C3 convertase and thereby blocks AP-dependent C3 activation and the amplification of CP- and LP-dependent C5 activation. Iptacopan does not, however, block the generation of MAC initiated by CP and LP. This is important, since it means that in immunized individuals, MAC-dependent killing of Neisseria species through activation of CP will be maintained.

### SUMMARY

The disclosure relates to methods of treating complement driven diseases, and in particular, lupus nephritis (LN) with iptacopan (Formula I, shown below) or a pharmaceutically acceptable salt thereof, e.g. iptacopan hydrochloride. Iptacopan is also known as LNP023. The terms “iptacopan” and “LNP023” are used herein interchangeably. Iptacopan (4-((2S,4S)-(4-ethoxy-1-((5-methoxy-7-methyl-1H-indol-4-yl)methyl) piperidin-2-yl))benzoic acid) belongs to the class of Factor B inhibitors of the complement pathway and acts by inhibiting or suppressing the amplification of the complement system caused by C3 activation irrespective of the initial mechanism of activation. Iptacopan hydrochloride is chemically designated as 4-((2S,4S)-(4-ethoxy-1-((5-methoxy-7-methyl-1H-indol-4-yl)methyl) piperidin-2-yl))benzoic acid hydrochloride of the following Formula (I):

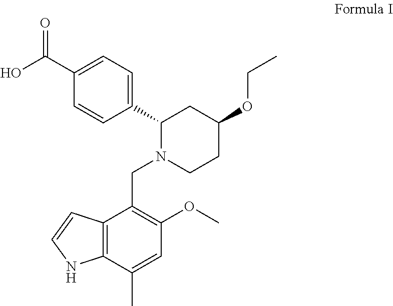 [SMILES: CCO[C@H]1CCN(Cc2c(OC)cc(C)c3[nH]ccc23)[C@H](c2ccc(C(=O)O)cc2)C1]

Iptacopan hydrochloride and methods for its preparation are disclosed in WO2015/009616 (see Example 26d), which is incorporated herein by reference in its entirety. The form of iptacopan hydrochloride used as the investigational study drug for this study is a monohydrate (Form H_{B}) as shown in the formula below:

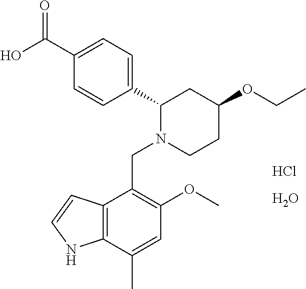 [SMILES: CCO[C@H]1CCN(Cc2c(OC)cc(C)c3[nH]ccc23)[C@H](c2ccc(C(=O)O)cc2)C1 ; Cl ; O]

### (2S,4S)-2-(4-Carboxyphenyl)-4-ethoxy-1-[(5-methoxy-7-methyl-1H-indol-4-yl)methyl]piperidin-1-ium chloride-water (1/1)

Iptacopan hydrochloride monohydrate Form H_{B} and methods for its preparation are disclosed in U.S. Ser. No. 63/026,637 and U.S. Ser. No. 63/052,699, published in WO 2021/234544, each of which is incorporated herein by reference in its entirety.

The disclosure provides a method of treating lupus nephritis (LN) in a subject, e.g., a patient, in need thereof, the method comprising orally administering to the subject, e.g., patient, iptacopan or a pharmaceutically acceptable salt thereof, e.g., iptacopan hydrochloride, at a dose of from about 50 mg to about 200 mg, e.g., from about 50 mg to from about 100 mg, from about 100 mg to about 200 mg, at a dose of about 50 mg, about 75 mg, about 100 mg, about 150 mg, or about 200 mg, each dose administered twice daily (b.i.d.), e.g., about every 12 hours, to thereby treat the subject, e.g., patient (wherein the dosing amount refers to the anhydrous free base of iptacopan hydrochloride), to thereby treat the subject, e.g., a patient.

### BRIEF DESCRIPTION OF THE DRAWINGS

FIG. 1 depicts a schematic of the study design.

### DETAILED DESCRIPTION

Described herein is the Phase 2 clinical study to determine safety and efficacy of iptacopan or a pharmaceutically acceptable salt thereof, e.g., iptacopan hydrochloride, in patients with lupus nephritis (LN), e.g., in addition to MMF, MPS or cyclophosphamide immunosuppressive treatment, both in combination with and as an alternative to a tapering corticosteroid regimen. Accordingly, described herein are methods of treating LN in a patient in need thereof, the method comprising orally administering, e.g., in capsule form, to the patient a twice daily dose, e.g., about every 12 hours, of iptacopan or a pharmaceutically acceptable salt thereof, e.g., iptacopan hydrochloride, (wherein the dosing amount refers to the anhydrous free base of iptacopan hydrochloride). Also described herein are methods of selecting the target patient population, methods of monitoring treatment of the target patient population, and methods of assessing safety and efficacy of treatment of the target patient population.

The details of the disclosure are set forth in the accompanying description below. Although methods and materials similar or equivalent to those described herein can be used in the practice or testing of the present disclosure, illustrative methods and materials are now described. Other features, objects, and advantages of the disclosure will be apparent from the description and from the claims. In the specification and the appended claims, the singular forms also include the plural unless the context clearly dictates otherwise. Unless defined otherwise, all technical and scientific terms used herein have the same meaning as commonly understood by one of ordinary skill in the art to which this disclosure belongs. All patents and publications cited in this specification are incorporated herein by reference in their entireties.

### Definitions

Unless specific definitions are provided, the nomenclature used in connection with, and the procedures and techniques of, analytical chemistry, synthetic organic chemistry, and medicinal and pharmaceutical chemistry described herein are those well known and commonly used in the art. Standard techniques may be used for chemical synthesis, and chemical analysis. Certain such techniques and procedures may be found for example in “Remington's Pharmaceutical Sciences,” Mack Publishing Co., Easton, Pa., 21st edition, 2005, which is hereby incorporated by reference for any purpose. Where permitted, all patents, applications, published applications and other publications and other data referred to throughout in the disclosure are incorporated by reference herein in their entirety.

Unless otherwise indicated, the following terms have the following meanings:

As used herein, “about” means within ±10% of a value.

As used herein, “administering” or “administration” means providing a pharmaceutical agent to an individual, and includes, but is not limited to, administering by a medical professional and self-administering. Administration of a pharmaceutical agent to an individual can be continuous, chronic, short or intermittent.

As used herein, the term “acquire” or “acquiring” as the terms are used herein, refer to obtaining possession of a physical entity (e.g., a sample, e.g., a blood sample or a blood plasma sample), or a value, e.g., a numerical value, by “directly acquiring” or “indirectly acquiring” the physical entity or value. “Directly acquiring” means performing a process (e.g., an analytical method) to obtain the physical entity or value. “Indirectly acquiring” refers to receiving the physical entity or value from another party or source (e.g., a third party laboratory that directly acquired the physical entity or value). Directly acquiring a value includes performing a process that includes a physical change in a sample or another substance, e.g., performing an analytical process which includes a physical change in a substance, e.g., a sample, performing an analytical method, e.g., a method as described herein, e.g., by sample analysis of bodily fluid, such as blood by, e.g., mass spectroscopy, e.g. LC-MS, e.g., LC-MS/MS methods.

As used herein, “baseline” refers to a time prior to treatment in relation to a characteristic of a subject or a patient.

As used herein, “dose” means a specified quantity of a pharmaceutical agent provided in a single administration, or in a specified time period. In certain embodiments, a dose can be administered in capsules. As used herein, the dosing amount refers to the anhydrous free base of iptacopan hydrochloride.

As used herein, “individual”, “patient”, “participant”, or “subject” means a human selected for treatment or therapy.

As used herein, “pharmaceutically acceptable salts” means physiologically and pharmaceutically acceptable salts of iptacopan, i.e., salts that retain the desired biological activity of iptacopan and do not impart undesired toxicological effects thereto. The term “pharmaceutically acceptable salt” or “salt” includes a salt prepared from pharmaceutically acceptable non-toxic acids or bases, including inorganic or organic acids and bases. “Pharmaceutically acceptable salts” of iptacopan may be prepared by methods well-known in the art. For a review of pharmaceutically acceptable salts, see Stahl and Wermuth, Handbook of Pharmaceutical Salts: Properties, Selection and Use (Wiley-VCH, Weinheim, Germany, 2002). Iptacopan hydrochloride and methods for its preparation are disclosed in WO2015/009616 (see Example 26d), which is incorporated herein by reference in its entirety.

As used herein, the term “treat” means decrease, suppress, attenuate, diminish, arrest, or stabilize the development or progression of a disorder or disease, e.g., lupus nephritis.

Unless otherwise specified, conventional definitions of terms control and conventional stable atom valences are presumed and achieved in all formulas and groups.

The articles “a” and “an” are used in this disclosure to refer to one or more than one (e.g., to at least one) of the grammatical object of the article. By way of example, “an element” means one element or more than one element.

### Methods of Use

Provided herein is a method of treating lupus nephritis (LN) in a subject, e.g., a patient, in need thereof, the method comprising administering, e.g., orally, to the subject, e.g., patient, iptacopan or a pharmaceutically acceptable salt thereof, e.g., iptacopan hydrochloride, at a dose of from about 50 mg to about 200 mg, e.g., from about 50 mg to from about 100 mg, from about 100 mg to about 200 mg, at a dose of about 50 mg, about 75 mg, about 100 mg, about 150 mg, or about 200 mg, each dose administered twice daily (b.i.d.), e.g., about every 12 hours (wherein the dosing amount refers to the anhydrous free base of iptacopan hydrochloride), to thereby treat the subject, e.g., patient.

Provided herein is a method of achieving complete renal response in a subject, e.g., a patient, in need thereof, the method comprising administering, e.g., orally, to the subject, e.g., patient, iptacopan or a pharmaceutically acceptable salt thereof, e.g., iptacopan hydrochloride, at a dose of from about 50 mg to about 200 mg, e.g., from about 50 mg to from about 100 mg, from about 100 mg to about 200 mg, at a dose of about 50 mg, about 75 mg, about 100 mg, about 150 mg, or about 200 mg, each dose administered twice daily (b.i.d.), e.g., about every 12 hours (wherein the dosing amount refers to the anhydrous free base of iptacopan hydrochloride), to thereby treat the subject, e.g., patient.

Provided herein is a method of reducing proteinuria in a subject, e.g., a patient, in need thereof, the method comprising administering, e.g., orally, to the subject, e.g., patient, iptacopan or a pharmaceutically acceptable salt thereof, e.g., iptacopan hydrochloride, at a dose of from about 50 mg to about 200 mg, e.g., from about 50 mg to from about 100 mg, from about 100 mg to about 200 mg, at a dose of about 50 mg, about 75 mg, about 100 mg, about 150 mg, or about 200 mg, each dose administered twice daily (b.i.d.), e.g., about every 12 hours (wherein the dosing amount refers to the anhydrous free base of iptacopan hydrochloride), to thereby treat the subject, e.g., patient.

In another aspect, the disclosure provides iptacopan or a pharmaceutically acceptable salt thereof, e.g., iptacopan hydrochloride, for use in the treatment of lupus nephritis (LN) in a subject, e.g., a patient, in need thereof, wherein the treatment comprises administering, e.g., orally, to the subject, e.g., patient, iptacopan or a pharmaceutically acceptable salt thereof, e.g., iptacopan hydrochloride, at a dose of from about 50 mg to about 200 mg, e.g., from about 50 mg to from about 100 mg, from about 100 mg to about 200 mg, at a dose of about 50 mg, about 75 mg, about 100 mg, about 150 mg, or about 200 mg, each dose to be administered twice daily (b.i.d.), e.g., about every 12 hours (wherein the dosing amount refers to the anhydrous free base of iptacopan hydrochloride).

In another aspect, the disclosure provides iptacopan or a pharmaceutically acceptable salt thereof, e.g., iptacopan hydrochloride, for use in achieving complete renal response in a subject, e.g., a patient, in need thereof, wherein the treatment comprises administering, e.g., orally, to the subject, e.g., patient, iptacopan or a pharmaceutically acceptable salt thereof, e.g., iptacopan hydrochloride, at a dose of from about 50 mg to about 200 mg, e.g., from about 50 mg to from about 100 mg, from about 100 mg to about 200 mg, at a dose of about 50 mg, about 75 mg, about 100 mg, about 150 mg, or about 200 mg, each dose to be administered twice daily (b.i.d.), e.g., about every 12 hours (wherein the dosing amount refers to the anhydrous free base of iptacopan hydrochloride). In an embodiment, the subject, e.g., patient, has or is diagnosed as having lupus nephritis.

In another aspect, the disclosure provides iptacopan or a pharmaceutically acceptable salt thereof, e.g., iptacopan hydrochloride, for use in reducing proteinuria in a subject, e.g., a patient, in need thereof, wherein the treatment comprises administering, e.g., orally, to the subject, e.g., patient, iptacopan or a pharmaceutically acceptable salt thereof, e.g., iptacopan hydrochloride, at a dose of from about 50 mg to about 200 mg, e.g., from about 50 mg to from about 100 mg, from about 100 mg to about 200 mg, at a dose of about 50 mg, about 75 mg, about 100 mg, about 150 mg, or about 200 mg, each dose to be administered twice daily (b.i.d.), e.g., about every 12 hours (wherein the dosing amount refers to the anhydrous free base of iptacopan hydrochloride). In an embodiment, the subject, e.g., patient, has or is diagnosed as having lupus nephritis.

In another aspect, the disclosure provides use of iptacopan or a pharmaceutically acceptable salt thereof, e.g., iptacopan hydrochloride, in the manufacture of a medicament for the treatment of lupus nephritis in a subject, e.g., a patient, in need thereof, wherein the treatment comprises orally administering to the subject, e.g., patient, iptacopan or a pharmaceutically acceptable salt thereof, e.g., iptacopan hydrochloride, at a dose of from about 50 mg to about 200 mg, e.g., from about 50 mg to from about 100 mg, from about 100 mg to about 200 mg, at a dose of about 50 mg, about 75 mg, about 100 mg, about 150 mg, or about 200 mg, each dose to be administered twice daily (b.i.d.), e.g., about every 12 hours (wherein the dosing amount refers to the anhydrous free base of iptacopan hydrochloride), to thereby treat the subject, e.g., patient. In an embodiment, the subject, e.g., patient, has or is diagnosed as having lupus nephritis.

In another aspect, the disclosure provides use of iptacopan or a pharmaceutically acceptable salt thereof, e.g., iptacopan hydrochloride, in the manufacture of a medicament for achieving complete renal response in a subject, e.g., a patient, in need thereof, wherein the treatment comprises orally administering to the subject, e.g., patient, iptacopan or a pharmaceutically acceptable salt thereof, e.g., iptacopan hydrochloride, at a dose of from about 50 mg to about 200 mg, e.g., from about 50 mg to from about 100 mg, from about 100 mg to about 200 mg, at a dose of about 50 mg, about 75 mg, about 100 mg, about 150 mg, or about 200 mg, each dose to be administered twice daily (b.i.d.), e.g., about every 12 hours (wherein the dosing amount refers to the anhydrous free base of iptacopan hydrochloride), to thereby treat the subject, e.g., patient. In an embodiment, the subject, e.g., patient, has or is diagnosed as having lupus nephritis.

In another aspect, the disclosure provides use of iptacopan or a pharmaceutically acceptable salt thereof, e.g., iptacopan hydrochloride, in the manufacture of a medicament for reducing proteinuria in a subject, e.g., a patient, in need thereof, wherein the treatment comprises orally administering to the subject, e.g., patient, iptacopan or a pharmaceutically acceptable salt thereof, e.g., iptacopan hydrochloride, at a dose of from about 50 mg to about 200 mg, e.g., from about 50 mg to from about 100 mg, from about 100 mg to about 200 mg, at a dose of about 50 mg, about 75 mg, about 100 mg, about 150 mg, or about 200 mg, each dose to be administered twice daily (b.i.d.), e.g., about every 12 hours (wherein the dosing amount refers to the anhydrous free base of iptacopan hydrochloride), to thereby treat the subject, e.g., patient. In an embodiment, the subject, e.g., patient, has or is diagnosed as having lupus nephritis.

In another aspect, the disclosure provides a pharmaceutical composition comprising iptacopan or a pharmaceutically acceptable salt thereof, e.g., iptacopan hydrochloride, for use in treating lupus nephritis, in a subject, e.g., patient, in need thereof, wherein the pharmaceutical composition is to be administered orally, at a dose of from about 50 mg to about 200 mg, e.g., from about 50 mg to from about 100 mg, from about 100 mg to about 200 mg, at a dose of about 50 mg, about 75 mg, about 100 mg, about 150 mg, or about 200 mg of iptacopan or a pharmaceutically acceptable salt thereof, e.g., iptacopan hydrochloride, each dose to be administered twice daily (b.i.d.), e.g., about every 12 hours (wherein the dosing amount refers to the anhydrous free base of iptacopan hydrochloride), to thereby treat the subject, e.g., patient.

In another aspect, the disclosure provides a pharmaceutical composition comprising iptacopan or a pharmaceutically acceptable salt thereof, e.g., iptacopan hydrochloride, for use in achieving complete renal response, in a subject, e.g., patient, in need thereof, wherein the pharmaceutical composition is to be administered orally, at a dose of from about 50 mg to about 200 mg, e.g., from about 50 mg to from about 100 mg, from about 100 mg to about 200 mg, at a dose of about 50 mg, about 75 mg, about 100 mg, about 150 mg, or about 200 mg of iptacopan or a pharmaceutically acceptable salt thereof, e.g., iptacopan hydrochloride, each dose to be administered twice daily (b.i.d.), e.g., about every 12 hours (wherein the dosing amount refers to the anhydrous free base of iptacopan hydrochloride), to thereby treat the subject, e.g., patient. In an embodiment, the subject, e.g., patient, has or is diagnosed as having lupus nephritis.

In another aspect, the disclosure provides a pharmaceutical composition comprising iptacopan or a pharmaceutically acceptable salt thereof, e.g., iptacopan hydrochloride, for use in reducing proteinuria, in a subject, e.g., patient, in need thereof, wherein the pharmaceutical composition is to be administered orally, at a dose of from about 50 mg to about 200 mg, e.g., from about 50 mg to from about 100 mg, from about 100 mg to about 200 mg, at a dose of about 50 mg, about 75 mg, about 100 mg, about 150 mg, or about 200 mg of iptacopan or a pharmaceutically acceptable salt thereof, e.g., iptacopan hydrochloride, each dose to be administered twice daily (b.i.d.), e.g., about every 12 hours (wherein the dosing amount refers to the anhydrous free base of iptacopan hydrochloride), to thereby treat the subject, e.g., patient. In an embodiment, the subject, e.g., patient, has or is diagnosed as having lupus nephritis.

The following embodiments apply to any of the foregoing aspects provided herein and may be combined in any order.

In an embodiment, the method or treatment comprises administering, e.g., orally, to the subject, e.g., patient, iptacopan hydrochloride monohydrate Form H_{B}.

In an embodiment, the subject, e.g., patient, has active glomerulonephritis ISN/RPS Class III or IV with or without co-existing class V features in renal biopsy.

In an embodiment, the subject, e.g., patient, has active Class III or IV lupus nephritis with or without co-existing features of Class V lupus nephritis in renal biopsy.

In an embodiment, the subject, e.g., patient, has an ANA titre≥1:80.

In an embodiment, the ANA titre is based on Hep-2 immunofluorescence assay or an equivalent positive enzyme immunoassay.

In an embodiment, the subject, e.g., patient, is positive for anti dsDNA.

In an embodiment, the subject, e.g., patient, has a urine protein/creatinine ratio (UPCR)≥1.5 g/g, prior to administering iptacopan or a pharmaceutically acceptable salt thereof.

In an embodiment, the urine protein/creatinine ratio is sampled from a first morning void or 24 hour urine collection.

In an embodiment, the subject, e.g., patient, has an estimated glomerular filtration rate (eGFR)≥30 ml/min/1.73 m², prior to administering iptacopan or a pharmaceutically acceptable salt thereof.

In an embodiment, the estimated glomerular filtration rate (eGFR) is calculated using the CKD-EPI formula or modified MDRD formula according to specific ethnic groups and local practice guidelines

In an embodiment, the subject, e.g., patient, has been vaccinated prior to treatment with iptacopan or a pharmaceutically acceptable salt thereof, e.g., iptacopan hydrochloride, prior to administering iptacopan or a pharmaceutically acceptable salt thereof.

In an embodiment, the subject, e.g., patient, has been vaccinated against Neisseria meningitidis (types A, C, Y and W-135) prior to administering iptacopan or a pharmaceutically acceptable salt thereof.

In an embodiment, the subject, e.g., patient, has been vaccinated against Streptococcus pneumoniae (Pneumovax-23) prior to administering iptacopan or a pharmaceutically acceptable salt thereof.

In an embodiment, the subject, e.g., patient, has been vaccinated against Haemophilus influenzae prior to administering iptacopan or a pharmaceutically acceptable salt thereof.

In an embodiment, the subject, e.g., patient, has been and is further being treated with supportive care, e.g., anti-malarials (e.g., hydroxychloroquine), ACEi, or ARB, e.g., at a maximal daily dose or maximally tolerated dose.

In an embodiment, the subject, e.g., patient, is further being treated with an agent.

In an embodiment, the agent is an immunosuppressant, e.g., mycophenolic acid (e.g., mycophenolate mofetil (MMF), mycophenolate sodium (MPS)), cyclophosphamide (CYC), an anti-B cell agent (e.g., belimumab, rituximab), a calcineurin inhibitor (e.g., voclosporin, cyclosporine A, tacrolimus).

In an embodiment, the subject, e.g., patient, is further being treated with a corticosteroid.

In an embodiment, the subject, e.g., patient, is further being treated with a corticosteroid according to a taper regimen.

As used herein “steroid tapering”, “taper”, “tapering regimen” and the like refer to a reduction regimen of a steroid (e.g., corticosteroid, e.g., glucocorticoid, e.g., prednisone, prednisolone, methylprednisolone) given to a patient over time. The tapering schedule (timing and dose decrease) will depend on the original steroid (e.g., corticosteroid, e.g., glucocorticoid, e.g., prednisone, prednisolone, methylprednisolone) dose the patient is taking prior to treatment with iptacopan. A tapering regimen is in alignment with common medical practice in SLE and is designed to minimize steroid related toxicity. Steroid tapering is a key goal to achieve in patients with SLE given that the current SoC SLE treatment regimens have substantial side effects from glucocorticoids and prolonged immunosuppression (Schwartz (2014). Curr Opin Rheumatol: 26: 502-509). In some embodiments of the disclosure, during treatment with iptacopan, the dose of steroid (e.g., corticosteroid, e.g., glucocorticoid, e.g., prednisone, prednisolone, methylprednisolone) administered to the patient is reduced using a taper regimen, and the patient does not experience a flare as a result of said reduction.

In some embodiments of the disclosure, when said method is used to treat a population of patients with LN, at least 50% of said patients achieve a daily steroid dose of <10 mg/day following a steroid tapering regimen during treatment with iptacopan.

In some embodiments of the disclosure, when said method is used to treat a population of patients with LN, at least 50% of said patients achieve a daily steroid dose of <5 mg/day following a steroid tapering regimen during treatment with iptacopan.

In some embodiments of the disclosure, when said method is used to treat a population of patients with LN, at least 50% of said patients achieve a daily steroid dose of <2.5 mg/day following a steroid tapering regimen during treatment with iptacopan.

In some embodiments of the disclosure, when said method is used to treat a population of patients with LN, at least 50% of said patients achieve a daily steroid dose of 0 mg/day following a steroid tapering regimen during treatment with iptacopan.

In some embodiments of the disclosure, when said method is used to treat a patient with LN, said patient achieves a daily steroid dose of <10 mg/day following a steroid tapering regimen during treatment with iptacopan.

In some embodiments of the disclosure, when said method is used to treat a patient with LN, said patient achieves a daily steroid dose of <5 mg/day following a steroid tapering regimen during treatment with iptacopan.

In some embodiments of the disclosure, when said method is used to treat a patient with LN, said patient achieves a daily steroid dose of <2.5 mg/day following a steroid tapering regimen during treatment with iptacopan.

In some embodiments of the disclosure, when said method is used to treat a patient with LN, said patient achieves a daily steroid dose of 0 mg/day following a steroid tapering regimen during treatment with iptacopan.

In an embodiment, the corticosteroid taper regimen is according to Table 1 below.

In an embodiment, the subject, e.g., patient, is further being treated with 2.5 mg daily steroid (e.g., corticosteroid, e.g., glucocorticoid, e.g., prednisone, prednisolone, methylprednisolone, prednisolone/prednisone equivalent) from Week 13 onwards.

In an embodiment, the subject, e.g., patient, is further being treated with 2.5 mg daily prednisolone/prednisone equivalent from Week 13 onwards.

In an embodiment, the subject, e.g., patient, is further being treated, with no more than 25 mg, e.g., no more than 20 mg, no more than 15 mg, no more than 10 mg, no more than 7.5 mg, no more than 5 mg, no more than 2.5 mg, at 25 mg, at 20 mg, at 15 mg, at 10 mg, at 7.5 mg, at 5 mg, or at 2.5 mg, daily steroid (e.g., corticosteroid, e.g., glucocorticoid, e.g., prednisone, prednisolone, methylprednisolone, prednisolone/prednisone equivalent), e.g., following a steroid tapering regimen.

TABLE 1
Guidance for IV (methylprednisolone or equivalent) and oral
corticosteroid (prednisone/prednisolone equivalent) taper

| Week | <45 kg | 45-65 kg | >65 kg |
|---|---|---|---|
| −2 weeks | 1000 mg IV methylprednisoline (or equivalent) | | |
| 0-2 | 25 | 35 | 40 |
| 3-4 | 20 | 25 | 30 |
| 5-6 | 15 | 20 | 30 |
| 6-7 | 10 | 15 | 15 |
| 7-8 | 7.5 | 10 | 10 |
| 9-10 | 7.5 | 7.5 | 7.5 |
| 11-12 | 5 | 5 | 5 |
| 13-14 | 2.5 | 2.5 | 2.5 |
| 14-15 | 2.5 | 2.5 | 2.5 |
| 15-16 | 2.5 | 2.5 | 2.5 |
| 17-18 | 2.5 | 2.5 | 2.5 |
| 19-20 | 2.5 | 2.5 | 2.5 |
| 21-24 | 2.5 | 2.5 | 2.5 |
| >24 | 2.5 | 2.5 | 2.5 |

In an embodiment, the subject, e.g., patient, is not further being treated with a steroid (e.g., corticosteroid, e.g., glucocorticoid, e.g., prednisone, prednisolone, methylprednisolone, prednisolone/prednisone equivalent), e.g., following a steroid tapering regimen.

In an embodiment, the subject, e.g., patient, is further being treated with at least one of an immunosuppressant and a corticosteroid.

In an embodiment, the subject, e.g., patient, is further being treated with an immunosuppressant, and a corticosteroid according to a taper regimen but without a corticosteroid following a steroid tapering regimen.

In an embodiment, the subject, e.g., patient, is further being treated an immunosuppressant without a steroid (e.g., corticosteroid, e.g., glucocorticoid, e.g., prednisone, prednisolone, methylprednisolone, prednisolone/prednisone equivalent).

In an embodiment, the subject, e.g., patient, is further being treated with an immunosuppressant and a steroid (e.g., corticosteroid, e.g., glucocorticoid, e.g., prednisone, prednisolone, methylprednisolone, prednisolone/prednisone equivalent), wherein the corticosteroid is administered according to a taper regimen.

In an embodiment, the subject, e.g., patient, is further being treated with an immunosuppressant, e.g., mycophenolic acid (e.g., mycophenolate mofetil (MMF), mycophenolate sodium (MPS)), cyclophosphamide (CYC), an anti-B cell agent (e.g., belimumab, rituximab), a calcineurin inhibitor (e.g., voclosporin, cyclosporine A, tacrolimus); and a steroid (e.g., corticosteroid, e.g., glucocorticoid, e.g., prednisone, prednisolone, methylprednisolone, prednisolone/prednisone equivalent), e.g., according to a taper regimen.

In an embodiment, the subject, e.g., patient, is further being treated with an immunosuppressant, e.g., mycophenolic acid (e.g., mycophenolate mofetil (MMF), mycophenolate sodium (MPS)), cyclophosphamide (CYC), an anti-B cell agent (e.g., belimumab, rituximab), a calcineurin inhibitor (e.g., voclosporin, cyclosporine A, tacrolimus); and a steroid (e.g., corticosteroid, e.g., glucocorticoid, e.g., prednisone, prednisolone, methylprednisolone, prednisolone/prednisone equivalent), e.g., with no more than 25 mg, e.g., no more than 20 mg, no more than 15 mg, no more than 10 mg, no more than 7.5 mg, no more than 5 mg, no more than 2.5 mg, at 25 mg, at 20 mg, at 15 mg, at 10 mg, at 7.5 mg, at 5 mg, or at 2.5 mg, daily steroid (e.g., corticosteroid, e.g., glucocorticoid, e.g., prednisone, prednisolone, methylprednisolone, prednisolone/prednisone equivalent), e.g., following a steroid tapering regimen.

In an embodiment, the subject, e.g., patient, is further being treated with an immunosuppressant, e.g., mycophenolate mofetil (MMF), mycophenolate sodium (MPS), cyclophosphamide (CYC), an anti-B cell agent (e.g., belimumab, rituximab), a calcineurin inhibitor (e.g., voclosporin, cyclosporine A, tacrolimus); without a corticosteroid, e.g., after a taper regimen.

In an embodiment, the subject, e.g., patient, is further being treated with an immunosuppressant, e.g., mycophenolate mofetil (MMF), mycophenolate sodium (MPS), cyclophosphamide (CYC), an anti-B cell agent (e.g., belimumab, rituximab), a calcineurin inhibitor (e.g., voclosporin, cyclosporine A, tacrolimus): without a corticosteroid.

In an embodiment, the subject, e.g., patient, is further being treated with MMF 1.5-3 g/day orally or MPS 1080 mg/day to 2160 mg/day orally initially during an initial treatment period, e.g., 6 months.

In an embodiment, the subject, e.g., patient, is further being treated with MMF 1.5-3 g/day orally or MPS 1080 mg/day to 2160 mg/day orally initially during an initial treatment period, e.g., 6 months, and titrated to a maximal tolerated dose.

In an embodiment, the subject, e.g., patient, is further being treated with MMF 1-2 g/day orally or MPS 720-1440 mg/day, after the initial treatment period, e.g., for 6 months.

In an embodiment, treating lupus nephritis (LN) comprises achieving an UPCR<0.5 g/g. e.g., sampled from a first morning void or 24 hour urine collection.

In an embodiment, the subject, e.g., patient, has an UPCR<0.5 g/g, e.g., sampled from a first morning void or 24 hour urine collection, in a subject, e.g., a patient, after administering iptacopan or a pharmaceutically acceptable salt thereof.

In an embodiment, treating lupus nephritis (LN) comprises achieving an UPCR in a subject, e.g., a patient, that is reduced, e.g., by no less than 15%, no less than 20%, no less than 25%, no less than 30%, no less than 40%, no less than 45%, no less than 50%, no less than 55%, no less than 60%, or no less than 65%, compared to prior to administering iptacopan or a pharmaceutically acceptable salt thereof.

In an embodiment, an UPCR in a subject, e.g., a patient, is reduced, e.g., by no less than 15%, no less than 20%, no less than 25%, no less than 30%, no less than 40%, no less than 45%, no less than 50%, no less than 55%, no less than 60%, or no less than 65%, compared to prior to administering iptacopan or a pharmaceutically acceptable salt thereof.

In an embodiment, treating lupus nephritis (LN) comprises achieving an eGFR≥90 mL/min/1.73 m², e.g., calculated using the CKD-EPI formula or modified MDRD formula according to specific ethnic groups and local practice guidelines.

In an embodiment, the subject, e.g., patient, has an eGFR≥90 mL/min/1.73 m², e.g., calculated using the CKD-EPI formula or modified MDRD formula according to specific ethnic groups and local practice guidelines, after administering iptacopan or a pharmaceutically acceptable salt thereof.

In an embodiment, treating lupus nephritis (LN) comprises achieving an eGFR that is increased, e.g., by from 2.5% to 5%, from 5% to 7.5%, from 7.5% to 10%, from 10% to 15%, or above 15%, compared to prior to administering iptacopan or a pharmaceutically acceptable salt thereof.

In an embodiment, the eGFR in a subject, e.g., a patient, is increased, e.g., by from 2.5% to 5%, from 5% to 7.5%, from 7.5% to 10%, from 10% to 15%, or above 15%, compared to prior to administering iptacopan or a pharmaceutically acceptable salt thereof.

In an embodiment, treating lupus nephritis (LN) comprises achieving an eGFR that is stable, e.g., no less than 80%, no less than 85%, no less than 90%, compared to prior to administering iptacopan or a pharmaceutically acceptable salt thereof.

In an embodiment, the eGFR in a subject, e.g., a patient, is stable, e.g., no less than 80%, no less than 85%, no less than 90%, compared to prior to administering iptacopan or a pharmaceutically acceptable salt thereof.

In an embodiment, treating lupus nephritis (LN) comprises achieving a complete renal response.

As used herein, the phrase “complete renal response” is defined as meeting the following criteria:
Estimated glomerular filtration rate (eGFR)≥90 mL/min/1.73 m² or no less than 85% of baseline value, and
24 h urine protein-to-creatinine ratio (UPCR)≤0.5 g/g.

In an embodiment, treating lupus nephritis (LN) comprises achieving a complete renal response without a renal flare.

As used herein, the phrase “renal flare” is defined as (Parikh et al 2014 Renal flare as a predictor of incident and progressive CKD in patients with lupus nephritis. Clin J Am Soc Nephrol p. 279-84: Yap et al 2017 Longterm Data on Disease Flares in Patients with Proliferative Lupus Nephritis in Recent Years. J Rheumatol p. 1375-1383; Ayoub et al 2019 Commentary on the Current Guidelines for the Diagnosis of Lupus Nephritis Flare. Curr Rheumatol Rep p. 12):
Proteinuric flare: an increase in UPCR (assessed from FMV) to over 1 g/g if patient had previously achieved CRR, or a doubling to greater than 2 g/g if patient had previously achieved PRR but not CRR.
Nephritic flare: a decrease in eGFR by ≥15% compared to stable level during remission.

In an embodiment, treating lupus nephritis (LN) comprises achieving a partial renal response.

As used herein, the phrase “partial renal response” is defined as meeting the following criteria:
Estimated glomerular filtration rate (eGFR)≥90 mL/min/1.73 m² or no less than 80% of baseline value, and
≥50% reduction in 24 h UPCR (compared to baseline) to <1 g/g (to <3 g/g if baseline≥3 g/g.

In an embodiment, treating lupus nephritis (LN) comprises achieving a partial renal response without a renal flare.

In an embodiment, a FACIT-Fatigue score of the subject, e.g., patient, is reduced compared to prior to administering iptacopan or a pharmaceutically acceptable salt thereof.

### Key Efficacy Assessment

Also provided herein is a method of assessing the efficacy of treatment in a patient population with lupus nephritis treated with iptacopan or a pharmaceutically acceptable salt thereof, e.g., iptacopan hydrochloride, at a dose of from about 50 mg to about 200 mg, e.g., from about 50 mg to from about 100 mg, from about 100 mg to about 200 mg, at a dose of about 50 mg, about 75 mg, about 100 mg, about 150 mg, or about 200 mg, each dose administered twice daily (b.i.d.), e.g., about every 12 hours, the method comprising determining the percentage of the patient population achieving complete renal response in the absence of renal flares as compared to a patient population untreated with iptacopan or a pharmaceutically acceptable salt thereof, e.g., iptacopan hydrochloride, to thereby assess efficacy of treatment.

Also provided herein is a method of assessing the efficacy of treatment in a patient population with lupus nephritis treated with iptacopan or a pharmaceutically acceptable salt thereof, e.g., iptacopan hydrochloride, at a dose of from about 50 mg to about 200 mg, e.g., from about 50 mg to from about 100 mg, from about 100 mg to about 200 mg, at a dose of about 50 mg, about 75 mg, about 100 mg, about 150 mg, or about 200 mg, each dose administered twice daily (b.i.d.), e.g., about every 12 hours, the method comprising determining the percentage of the patient population achieving partial renal response in the absence of renal flares as compared to a patient population untreated with iptacopan or a pharmaceutically acceptable salt thereof, e.g., iptacopan hydrochloride, to thereby assess efficacy of treatment.
Patient population: defined through appropriate inclusion/exclusion criteria to reflect the targeted LN population.
Proportion of patients achieving Complete Renal Response (CRR) in the absence of renal flares. CRR is defined as meeting the following criteria:
   Estimated glomerular filtration rate (eGFR)≥90 mL/min/1.73 m² or no less than 85% of baseline value, and
   24 h urine protein-to-creatinine ratio (UPCR)≤0.5 g/g
Proportion of patients achieving partial renal response (PRR) in the absence of renal flares. PRR is defined as meeting the following criteria:
   Estimated glomerular filtration rate (eGFR)≥90 mL/min/1.73 m² or no less than 80% of baseline value, and
   ≥50% reduction in 24 h UPCR (compared to baseline) to <1 g/g (to <3 g/g if baseline≥3 g/g

The average of two 24 h UPCR values will be used to derive CRR/PRR, both collected within 10 days before the respective study visit.
Renal flares in patients who have achieved complete or partial renal response (CRR or PRR) are objectively defined as (Parikh et al 2014 Renal flare as a predictor of incident and progressive CKD in patients with lupus nephritis. Clin J Am Soc Nephrol p. 279-84: Yap et al 2017 Longterm Data on Disease Flares in Patients with Proliferative Lupus Nephritis in Recent Years. J Rheumatol p. 1375-1383; Ayoub et al 2019 Commentary on the Current Guidelines for the Diagnosis of Lupus Nephritis Flare. Curr Rheumatol Rep p. 12):
   Proteinuric flare: an increase in UPCR (assessed from FMV) to over 1 g/g if patient had previously achieved CRR, or a doubling to greater than 2 g/g if patient had previously achieved PRR but not CRR.
   Nephritic flare: a decrease in eGFR by ≥15% compared to stable level during remission.

### EXAMPLES

The disclosure is further illustrated by the following examples and synthesis schemes, which are not to be construed as limiting this disclosure in scope or spirit to the specific procedures herein described. It is to be understood that the examples are provided to illustrate certain embodiments and that no limitation to the scope of the disclosure is intended thereby. It is to be further understood that resort may be had to various other embodiments, modifications, and equivalents thereof which may suggest themselves to those skilled in the art without departing from the spirit of the present disclosure and/or scope of the appended claims.

List of abbreviations

| | |
|---|---|
| AE | Adverse Event |
| b.i.d. | bis in die/twice a day |
| CNI | Calcineurin inhibitor |
| CP | Complement Pathway |
| CRF | Case Report/Record Form (paper or electronic) |
| EDC | Electronic Data Capture |
| eGFR | Estimated glomerular filtration rate |
| i.V. | intravenous |
| IN | Investigator Notification |
| IVH | Intravascular Hemolysis |
| LDH | lactate dehydrogenase |
| MAVE | Major Adverse Vascular Event |
| mg | milligram(s) |
| mL | milliliter(s) |
| p.o. | oral(ly) |
| PD | Pharmacodynamic(s) |
| PK | Pharmacokinetic(s) |
| PT | prothrombin time |
| QD | Once a day |
| RBC | red blood cell(s) |
| SAE | Serious Adverse Event |
| ULN | upper limit of normal |
| UPCR | urine protein/creatinine ratio |
| WBC | white blood cell(s) |

Glossary of terms

| | |
|---|---|
| Additional | Medicinal products that may be used during the clinical trial as |
| treatment | described in the protocol, but not as an investigational |
| | medicinal product (e.g. any background therapy) |
| Assessment | A procedure used to generate data required by the study |
| Biologic Samples | A biological specimen including, for example, blood (plasma, |
| | serum), saliva, tissue, urine, stool, etc. taken from a study |
| | participant |
| Cohort | A specific group of participants fulfilling certain criteria and |
| | generally treated at the same time |
| Control drug | A study drug (active or placebo) used as a comparator to reduce |
| | assessment bias, preserve blinding of investigational drug, |
| | assess internal study validity, and/or evaluate comparative |
| | effects of the investigational drug |
| Dosage | Dose of the study treatment given to the participant in a time |
| | unit (e.g. 100 mg once a day, 75 mg twice a day) |
| Electronic Data | Electronic data capture (EDC) is the electronic acquisition of |
| Capture (EDC) | clinical study data using data collection systems, such as Web- |
| | based applications, interactive voice response systems and |
| | clinical laboratory interfaces. EDC includes the use of |
| | Electronic Case Report Forms (eCRFs) which are used to |
| | capture data transcribed from paper source forms used at the |
| | point of care |
| End of the clinical | The end of the clinical trial is defined as the last visit of the last |
| trial | participant or at a later point in time as defined by the protocol |
| Enrollment | Point/time of participant entry into the study at which informed |
| | consent must be obtained |
| Estimand | A precise description of the treatment effect reflecting the |
| | clinical question posed by the trial objective. It summarizes at |
| | a population-level what the outcomes would be in the same |
| | patients under different treatment conditions being compared. |
| | Attributes of an estimand include the population, variable (or |
| | endpoint) and treatment of interest, as well as the specification |
| | of how the remaining intercurrent events are addressed and a |
| | population-level summary for the variable. |
| Healthy volunteer | A person with no known significant health problems who |
| | volunteers to be a study participant |
| Intercurrent events | Events occurring after treatment initiation that affect either the |
| | interpretation or the existence of the measurements associated |
| | with the clinical question of interest. |
| Investigational | The drug whose properties are being tested in the study |
| drug/ treatment | |
| Medication | A unique identifier on the label of medication kits |
| number | |
| Mis-randomized | Mis-randomized participants are those who were not qualified |
| participants | for randomization and who did not take study treatment, but |
| | have been inadvertently randomized into the study |
| Other treatment | Treatment that may be needed/allowed during the conduct of |
| | the study (i.e. concomitant or rescue therapy) |
| Part | A sub-division of a study used to evaluate specific objectives or |
| | contain different populations. For example, one study could |
| | contain a single dose part and a multiple dose part, or a part in |
| | participants with established disease and in those with newly- |
| | diagnosed disease |
| Participant | A trial participant (can be a healthy volunteer or a patient) |
| Participant number | A unique number assigned to each participant upon signing the |
| | informed consent. This number is the definitive, unique |
| | identifier for the participant and should be used to identify the |
| | participant throughout the study for all data collected, sample |
| | labels, etc. |
| Period | The subdivisions of the trial design (e.g. Screening, Treatment, |
| | Follow-up) which are described in the Protocol. Periods define |
| | the study phases and will be used in clinical trial database setup |
| | and eventually in analysis |
| Personal data | Participant information collected by the Investigator that is |
| | coded and transferred to Novartis for the purpose of the clinical |
| | trial. This data includes participant identifier information, study |
| | information and biological samples. |
| Premature | Point/time when the participant exits from the study prior to the |
| participant | planned completion of all study drug administration and/or |
| withdrawal | assessments; at this time all study drug administration is |
| | discontinued and no further assessments are planned |
| Randomization | A unique identifier assigned to each randomized participant |
| number | |
| Screen Failure | A participant who did not meet one or more criteria that were |
| | required for participation in the study |
| Source | Source data refers to the initial record, document, or primary |
| Data/Document | location from where data comes. The data source can be a |
| | database, a dataset, a spreadsheet or even hard-coded data, such |
| | as paper or eSource |
| Start of the clinical | The start of the clinical trial is defined as the signature of the |
| trial | informed consent by the first participant |
| Study treatment | Any drug or combination of drugs or intervention administered |
| | to the study participants as part of the required study |
| | procedures; includes investigational drug(s), control(s) or |
| | background therapy |
| Study treatment | When the participant permanently stops taking any of the study |
| discontinuation | drug(s) prior to the defined study treatment completion date (if |
| | any) for any reason; may or may not also be the point/time of |
| | study discontinuation |
| Treatment | A treatment arm/group defines the dose and regimen or the |
| arm/group | combination, and may consist of 1 or more cohorts. |
| Treatment of | The treatment of interest and, as appropriate, the alternative |
| interest | treatment to which comparison will be made. These might be |
| | individual interventions, combinations of interventions |
| | administered concurrently, e.g. as add-on to standard of care, or |
| | might consist of an overall regimen involving a complex |
| | sequence of interventions. This is the treatment of interest used |
| | in describing the related clinical question of interest, which |
| | might or might not be the same as the study treatment. |
| Variable (or | The variable (or endpoint) to be obtained for each participant |
| endpoint) | that is required to address the clinical question. The |
| | specification of the variable might include whether the |
| | participant experiences an intercurrent event. |
| Withdrawal of | Withdrawal of consent from the study occurs only when a |
| study consent | participant does not want to participate in the study any longer |
| (WoC) | and does not allow any further collection of personal data |

### Example 1. An Adaptive, Randomized, Double-Blind, Dose Exploration, Parallel Group, Placebo Controlled, Multicenter Phase 2 Trial to Evaluate the Efficacy, Safety and Tolerability of LNP023 in Combination with Standard-of-Care with and without Oral Corticosteroids in Patients with Active Lupus Nephritis Class III-IV, +/−V

### Purpose

The overall purpose of this two-part study is to evaluate the efficacy, safety and tolerability of iptacopan (LNP023) in addition to MMF/MPS immunosuppressive treatment both in combination with and as an alternative to a tapering corticosteroid regimen in patients with active LN (ISN/RPS Class III or IV, with or without co-existing class V features).

Iptacopan will be first evaluated in a proof-of-concept part at a dose of 200 mg b.i.d. as an add-on to SoC (corticosteroid tapering+MMF/MPS) with respect to inducing a clinically meaningful increase in complete renal response (CRR) and reduction in proteinuria compared to SoC (Part 1). The data collected in this proof-of-concept part will determine whether to start Part 2 of the study, where iptacopan will be evaluated at a lower dose of 50 mg b.i.d. on top of SoC, and with the previously tested dose of 200 mg b.i.d. as an alternative to corticosteroids as LN patients would benefit from an effective, steroid-free therapy.

### Primary Objectives and Endpoints

The primary objectives are:
Four treatment schemes are considered:
(a) iptacopan 200 mg+corticosteroid taper+MMF/MPS (“iptacopan 200 mg+steroid”)
(b) iptacopan 50 mg+corticosteroid taper+MMF/MPS (“iptacopan 50 mg+steroid”)
(c) iptacopan 200 mg+no corticosteroid taper+MMF/MPS (“iptacopan 200 mg alone”)
(d) no iptacopan+corticosteroid taper+MMF/MPS (“steroid alone”)
Part 1:
   i) To evaluate the proportion of patients achieving complete renal response (CRR) at week 24 with treatment (a) “iptacopan 200 mg+steroid” compared to (d) “steroid alone”
Part 2*:
   ii) To evaluate the proportion of patients achieving complete renal response (CRR) at week 24 with treatment (b) “iptacopan 50 mg+steroid” compared to (d) “steroid alone”
   iii) To evaluate the proportion of patients achieving complete renal response (CRR) at week 24 with treatment (c) “iptacopan 200 mg alone” compared to (d) “steroid alone”*Due to the adaptive nature of the study the comparisons (ii) and (iii) will be performed only if Part 2 is started.

### Secondary Objectives:

Parts 1 and 2:
To evaluate the superiority of:
(i) treatment “iptacopan 200 mg+steroid” compared to “steroid alone”, and of
(ii) treatment “iptacopan 50 mg+steroid” compared to “steroid alone”, and of
(iii) treatment “iptacopan 200 mg alone” compared to “steroid alone”
   in achieving CRR or PRR at week 24
   in achieving CRR at week 52
   in achieving early CRR
   in achieving CRR or PRR rate at week 52
   in achieving at least 25% improvement in 24 h UPCR at week 24
   in reducing the incidence of renal flares between week 24 and week 52
   in reducing the frequency of courses of corticosteroids for renal and non-renal indications at a dose exceeding an average of 20 mg/day (of prednisolone or equivalent) for more than 10 days at week 24 and week 52
in change from baseline in FACIT-Fatigue score at week 24 and week 52
   in change from baseline in SLEDAI-2K score at week 24 and week 52
   in change from baseline in BILAG-2004 score at weeks 24 and 52
   To evaluate the safety and tolerability of 52 weeks of treatment with “iptacopan 200 mg+steroid”, “iptacopan 50 mg+steroid”, and “iptacopan 200 mg alone” compared to “steroid alone”
   To assess the dose-exposure response for reduction in proteinuria at week 24 to iptacopan on top of SoC (corticosteroid tapering+MMF/MPS)

### Study Design

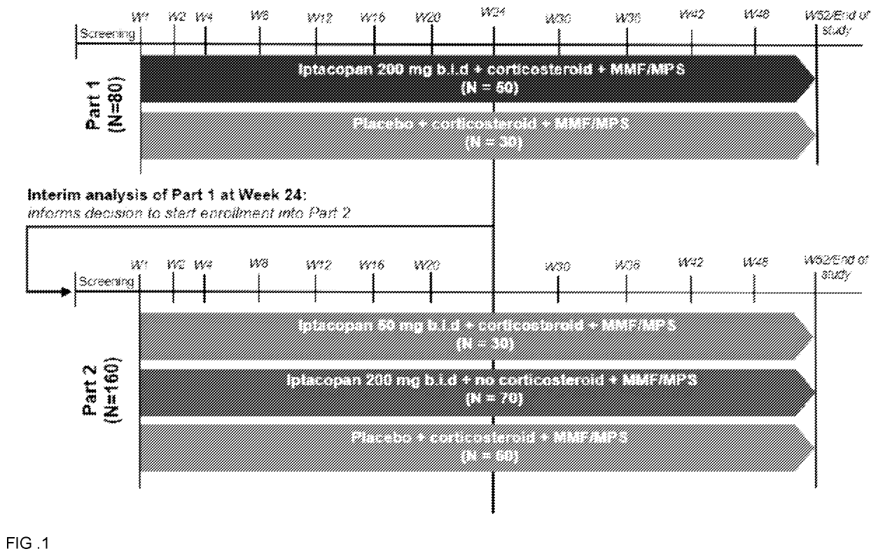

This study is a two-part, dose exploration, adaptive, randomized, double-blind, parallel group, placebo-controlled multi-centre study evaluating the efficacy, safety and tolerability of iptacopan 50 mg b.i.d and 200 mg b.i.d in combination with MMF/MPS for the treatment of LN. (see FIG. 1):
Part 1 will evaluate whether the use of iptacopan 200 mg b.i.d. is efficacious and safe as an add-on therapy to MMF/MPS plus a tapering corticosteroid regimen. Approximately 80 patients will be randomized in a 5:3 ratio to each treatment arm to ensure adequate power at the interim analysis. Part 1 is comprised of a screening period which can last up to a maximum of 6 weeks. A pre-specified interim analysis (IA) will be performed at the time when approximately all enrolled patients have completed the week 24 visit. The purpose of the IA is to assess the effect of iptacopan 200 mg b.i.d.+MMF/MPS+corticosteroid taper versus matching placebo+MMF/MPS+tapering corticosteroid regimen on the complete renal response (CRR) and reduction in proteinuria assessed by the UPCR value, both determined from two 24 h urine collections. The totality of efficacy and safety data up to the interim analysis cut-off point and the outcome of the prespecified IA will determine the start of the Part 2 of the study. The double-blind treatment period will continue up to week 52 for Part 1.
Part 2 will begin upon confirmation of a positive result from IA. Part 2 will evaluate whether (i) the use of iptacopan 50 mg b.i.d. is efficacious and safe as an add-on therapy to MMF/MPS plus a tapering corticosteroid regimen, and (ii) the use of iptacopan 200 mg b.i.d. is efficacious and safe as an add-on therapy to MMF/MPS without the use of corticosteroids versus iptacopan matching placebo+MMF/MPS+corticosteroid tapering regimen. After confirmation of eligibility criteria, approximately 160 patients will be randomized in a 3:7:6 ratio respectively to one of the three treatment arms to ensure adequate power for all comparisons in the primary analysis. The active treatment in the control arm is the same as in Part 1 and the control patients from both parts will be pooled in the primary analysis to achieve greater power. Part 2 is comprised of a screening period which can last up to a maximum of 6 weeks. The double-blind treatment period will continue up to week 52 for Part 2.

### Study Design Rationale

This study is a two-part, adaptive, dose exploration, randomized, double-blind, parallel group, placebo-controlled multi-center study evaluating the efficacy and safety of iptacopan in addition to mycophenolate mofetil (MMF) or mycophenolate sodium (MPS) immunosuppressive treatment both in combination with and as an alternative to corticosteroids in patients with active LN (ISN/RPS Class III or IV, with or without co-existing class V features).

### Study Population

The study population will be comprised of 240 randomized participants consisting of male and female patients aged 18 years or older with LN, having undergone a renal biopsy within 3 months prior to screening showing ISN/RPS Class III or IV LN with or without co-existing Class V features. A repeat renal biopsy will be needed to verify LN as a main cause of the flare and exclude other factors such as scarring, infection and drug toxicity if more than 3 months has elapsed since the last biopsy. Patients enrolled into both Part 1 and Part 2 of this trial should have evidence of an active nephritis, as defined by a UPCR (based on a 24 h urine collection) of ≥1.5 g/g (confirmed at 2 separate or consecutive visits), a positive dipstick for hematuria (not associated with menstruation or UTI) requiring treatment with MPA-based immunosuppressive regimen according to local SoC.

### Inclusion Criteria

Participants eligible for inclusion in this study must meet all of the following criteria. All criteria noted below are for both Part 1 and Part 2 unless otherwise specified.
1. Adult male and female patients aged 18 years old or older at the time of screening
2. Signed informed consent must be obtained prior to participation in the study: patients should be able to communicate well with the investigator, understand and comply with the requirements of the study
3. Unequivocally positive ANA test result defined as an ANA titre≥1:80 (based on HEp-2 immunofluorescence assay or an equivalent positive enzyme immunoassay) and/or a positive anti dsDNA at screening
4. Active biopsy-proven lupus nephritis within 3 months of screening demonstrating Class III or IV lupus nephritis with or without co-existing features of Class V lupus nephritis. If a biopsy was not performed within 3 months of screening, a repeat biopsy is needed to verify LN as a main cause of flare. The renal biopsy will need to be performed during the screening period and after confirmation that the patient has met all other inclusion/exclusion criteria
5. Documentation of active renal disease at the time of screening necessitating the commencement of therapy with corticosteroids in combination with MMF/MPS. Active renal disease will be defined by the following:
   Positive dipstick for hematuria (not associated with menstruation or UTI)
   Proteinuria (to be confirmed at screening and prior to randomization)
      At screening: UPCR of ≥1.5 g/g sampled from a first morning void or 24 hour urine collection
      Prior to randomization: Confirmation of UPCR≥1.5 g/g sampled from a 24 hour urine collection on two separate days, within a window of 10 days prior to randomization
6. eGFR≥30 ml/min/1.73 m² (eGFR calculated using the CKD-EPI formula or modified MDRD formula according to specific ethnic groups and local practice guidelines)
7. Vaccination against Neisseria meningitidis and Streptococcus pneumoniae infections is required prior to the start of study treatment. If the patient has not been previously vaccinated, or if a booster is required, vaccine should be given according to local regulations at least 2 weeks prior to first study drug administration. If study treatment is expected to start earlier than 2 weeks post vaccination, prophylactic antibiotic treatment should be given for at least 2 weeks after vaccination.
8. Vaccination against Haemophilus influenzae infection should be given, if available and according to local regulations, at least 2 weeks prior to first study drug administration. If study treatment is expected to start earlier than 2 weeks post vaccination, prophylactic antibiotic treatment should be given at the start of study treatment and continue for at least 2 weeks after vaccination.
9. All patients should have been on supportive care including stable dose regimen of antimalarials (e.g. hydroxychloroquine) unless contraindicated, ACEi or ARB at either locally approved maximal daily dose or the maximally tolerated dose (per investigators' judgement) at screening, as per the local clinical practice. Doses should remain stable throughout the study. First presentation or flare of lupus nephritis. All participants with a LN flare, following prior treatment with cyclophosphamide can be included. Participants who have developed a LN flare following treatment with MMF may be included if the treating physician is of the opinion that participation in the study is of potential benefit to the patient, considering the doses of MMF with or without corticosteroids being used in the study protocol
10. All participants must be vaccinated for COVID-19 prior to randomization as per local SoC. Participants should also have a COVID-19 test performed as per local SoC.

### Key Exclusion Criteria

Participants meeting any of the following criteria are not eligible for inclusion in this study. All criteria noted below are for both Part 1 and Part 2 unless otherwise specified
1. Participants who in the opinion of the investigator have previously failed to respond to therapy with MMF/MPS will not be included.
2. Induction treatment with cyclophosphamide within 3 months of planned treatment for this study: treatment with calcineurin inhibitors within the previous 3 months prior to screening
3. Presence of rapidly progressive glomerulonephritis (RPGN) as defined by 50% decline in eGFR within 3 months prior to screening.
4. Renal biopsy presenting with interstitial fibrosis/tubular atrophy (IF/TA) or glomerulosclerosis of more than 50%, or which in the opinion of the investigator is such that it precludes likely response to immunosuppressive therapy.
5. Patients previously treated with immunosuppressive or other immunomodulatory agents which are not considered standard of care for treatment of lupus nephritis within the previous 1 year
6. Participants being treated with systemic corticosteroids (>5 mg/day prednisone or equivalent) for indications other than SLE or LN e.g. acute asthma, inflammatory bowel disease.
7. Participants being treated with systemic corticosteroids for SLE or LN will be excluded if they have taken more than an average of 10 mg/day prednisone (or equivalent) in the previous 4 weeks and more than an average of 20 mg/day in the previous 1 week
8. Receipt of more than a total dose of 1000 mg equivalent i.v. pulse methylprednisolone (cumulative dose) within 2 weeks prior to enrollment (and at enrollment)
9. Prior treatment with any of the following within 1 year prior to screening:
   Nitrogen mustard, chlorambucil, vincristine, procarbazine, etoposide, abatacept,
   Treatment with any B-cell targeted therapy
   Treatment with biological investigational agent
   Treatment with interleukin-6 targeted therapy
10. Participants with current clinical, radiographic, or laboratory evidence of active or latent TB: history of active TB within 2 years of screening (even if treated); in the opinion of the investigator and based on appropriate evaluation, have a risk of reactivation of TB that precludes the use of conventional immunosuppression

### Study Treatment

In Part 1, study participants will be randomized to receive iptacopan 200 mg b.i.d. as an add-on to SoC (MMF/MPS+CS).

In Part 2, study participants will be randomized to receive either (i) 50 mg b.i.d. as an add-on to SoC for LN (MMF/MPS+CS) or (ii) iptacopan 200 mg b.i.d.+MMF/MPS without corticosteroids.

### Initial Therapy

The recommended doses of MMF/MPS for initial therapy are as follows: MMF 1.5-3 g/day orally or MPS 1080 mg/day to 2160 mg/day orally. The investigator should determine the appropriate starting dose for an individual participant based on the consideration of clinical benefit and side-effects that may be expected. However, it is expected that the dose is titrated to the maximal tolerated dose within the recommended dose range by week 2. At the time of study entry and confirmation of eligibility, patients should be started on the recommended dose of MMF or MPS plus iptacopan/matching placebo and commence the tapering corticosteroid regimen/matching placebo. A reduction of MMF/MPS dose is only allowed in case of toxicity or intolerance and as per investigators discretion. As per guidelines, after 6 months of initial therapy with MMF or MPS, the dose of MMF may be reduced or switched per investigator discretion and/or local SoC.

Intraveneous corticosteroids must be given to all patients in all treatment arms at a cumulative total dose of no more than 1000 mg i.v. methylprednisolone or equivalent within 2 weeks of randomization and prior to commencement of the oral corticosteroid tapering regimen. Oral corticosteroids will be permitted prior to enrollment at a prednisolone/prednisone dose of no more than an average of 10 mg/day, or equivalent in the previous 4 weeks (does not include prior i.v. corticosteroid dose) and no more than an average of 20 mg/day in the previous 1 week. Forced tapering with oral corticosteroids will be started on day 1 at a starting dose dependent on weight as shown in Table 1. The predefined, guided corticosteroid taper regimen must be followed (see Table 1). From Week 13 onwards, the target dose of oral corticosteroids for all participants is 2.5 mg daily prednisolone/prednisone equivalent.

### Maintenance Therapy

After the end of the initial treatment period and if determined by investigator, the dose of MMF may be reduced to 1-2 g/day (MPS to 720-1440 mg/day) in accordance with local standard of care.

### Treatment Duration

The planned duration of Part 1 and Part 2 is 52 weeks (for each part). Participants may be discontinued from treatment early due to unacceptable toxicity (reported as AEs), disease progression and/or at the discretion of the investigator or participant.

### Dose and Duration of Treatment Rationale

The study will first investigate the treatment effect of iptacopan 200 mg b.i.d. on top of SoC compared to SoC alone (Part 1). This dose was chosen based on the least sensitive alternative pathway biomarker (Wieslab assay); it ensures >90% target inhibition in more than 70% of individuals, as determined from FIH trial CLNP023X2101. Dose range finding with doses between 10 to 200 mg b.i.d was performed in Phase 2 studies in IgAN, C3G and PNH. Based on an integrated assessment of key biomarkers, efficacy and safety endpoints, the 200 mg b.i.d. dose was selected as it showed the greatest therapeutic potential:
In the healthy volunteer studies (CLNP023X2101 and CLNP023X1102), iptacopan was administered to 102 subjects in single ascending dose (SAD, 10 to 400 mg) and multiple ascending dose (MAD, 10 to 200 mg b.i.d. for 2 weeks). The results showed that iptacopan was well tolerated, had high solubility, good permeability, and a fast absorption. No deaths, serious adverse events (SAEs), or adverse events (AEs) leading to study drug discontinuation were observed.
Preliminary data from ongoing Phase 2 studies in PNH show that PNH patients treated with iptacopan 200 mg b.i.d., both as an add-on therapy to eculizumab (CLNP023X2201) and as a monotherapy (CLNP023X2204), have clinically relevant benefits: the majority of patients showed a reduction in LDH levels and improvements in hemoglobin in the absence of red blood cell (RBC) transfusions. This was achieved through control of both intra- and extravascular hemolysis. In addition, iptacopan was overall safe and well tolerated in both studies.
Interim analysis results from the ongoing Phase 2 study in patients with IgAN (CLNP023X2203) showed a statistically significant dose-response effect of iptacopan on UPCR at 90 days, which was the primary endpoint of this study. A clinically meaningful UPCR reduction and stable/improving eGFR levels were observed for the 200 mg b.i.d. dose compared to placebo. Iptacopan demonstrated an overall well-tolerated safety profile.
Interim analysis results from a Phase 2 study in patients with C3G (CLNP023X2202) showed that treatment with iptacopan at 200 mg b.i.d. was also well-tolerated by these patients and associated with a 49% reduction in UPCR and stabilization of eGFR at 12 weeks.

If iptacopan 200 mg b.i.d. shows a clinically meaningful effect on top of SoC in Part 1 (evaluated in the interim analysis on Week 24 or in a repeated interim analysis on Week 52), a lower dose of 50 mg b.i.d. on top of SoC will be explored in Part 2. The dose finding studies described above suggest that the 50 mg b.i.d. dose may lie within the dynamic range of the dose-exposure relationship for the key biomarkers, UPCR, and eGFR. Together with the 200 mg b.i.d. dose, 50 mg b.i.d. will inform about a wide range of the dose-exposure relationship in lupus nephritis.

### Efficacy

Complete Renal Response (CRR)
Partial Renal Response (PRR)
Proteinuria quantified by the Urine Protein-to-Creatinine Ratio (UPCR)
Estimated Glomerular Filtration Rate (eGFR)
Renal flares
British Isles Lupus Activity (BILAG) score (BILAG-2004)
Systemic Lupus Erythematosus Disease Activity Index 2000 (SLEDAI-2K)
Functional Assessment of Chronic Illness Therapy Fatigue (FACIT-Fatigue)
Corticosteroid dosing

### EQUIVALENTS

Those skilled in the art will recognize, or be able to ascertain, using no more than routine experimentation, numerous equivalents to the specific embodiments described specifically herein. Such equivalents are intended to be encompassed in the scope of the following claims.

1. A method of treating lupus nephritis (LN) in a subject in need thereof, the method comprising orally administering to the subject iptacopan or a pharmaceutically acceptable salt thereof, at a dose of from about 50 mg to about 200 mg administered twice daily (b.i.d.), to thereby treat the subject.

2. The method of claim 1, wherein the dose is about 50 mg twice daily.

3. The method of claim 1, wherein the dose is about 100 mg twice daily.

4. The method of claim 1, wherein the dose is about 200 mg twice daily.

5. The method of claim 1, wherein the method further comprising administering to the subject an immunosuppressant.

6. The method of claim 5, wherein the immunosuppressant is a mycophenolic acid, a cyclophosphamide (CYC), an anti-B cell agent, or a calcineurin inhibitor (CNI).

7. The method of claim 1, wherein the method further comprising administering to the subject a corticosteroid.

8. The method of claim 7, wherein the corticosteroid is administered to the subject according to a tapering regimen.

9. The method of claim 1, wherein the subject achieves a daily corticosteroid dose of <10 mg/day following a corticosteroid tapering regimen.

10. The method of claim 1, wherein the subject achieves a daily corticosteroid dose of 0 mg/day following a corticosteroid d tapering regimen.

11. The method of claim 1, wherein the immunosuppressant is administered without a corticosteroid.

12. The method of claim 1, wherein the subject has been vaccinated prior to administration of iptacopan or a pharmaceutically acceptable salt thereof against Neisseria meningitidis and Streptococcus pneumoniae.

13. The method of claim 1, wherein the subject has been vaccinated prior to administration of iptacopan or a pharmaceutically acceptable salt thereof against Haemophilus influenzae.

14. The method of claim 1, wherein the subject has been further treated with supportive care, wherein the supportive care is selected from the group consisting of an antimalarial (e.g. hydroxychloroquine), an angiotensin-converting enzyme inhibitor and an angiotensin receptor blocker.

15. The method of claim 1, wherein treating lupus nephritis (LN) comprises achieving an UPCR<0.5 g/g in a subject.

16. The method of claim 1, wherein treating lupus nephritis (LN) comprises achieving an UPCR in a subject that is reduced by 50% compared to prior to administering iptacopan or a pharmaceutically acceptable salt thereof, e.g., the UPCR value being measured by sampling from a first morning void or 24 hour urine collection.

17. The method of claim 1, wherein treating lupus nephritis (LN) comprises achieving an eGFR≥90 ml/min/1.73 m².

18. The method of claim 1, wherein treating lupus nephritis (LN) comprises achieving an eGFR value of no less than 80% (e.g., no less than 85%) compared to prior to administering iptacopan or a pharmaceutically acceptable salt thereof.

19-20. (canceled)
